(19) United States
(12) Patent Application Publication
(10) Pub. No.: US 2025/0066273 A1
(43) Pub. Date: Feb. 27, 2025

(54) PROCESS FOR SEPARATING AN OLEFIN STREAM FROM METHANE

(71) Applicant: UOP LLC, Des Plaines, IL (US)

(72) Inventors: Ian G. Horn, Streamwood, IL (US); Andrew B. Chin, Wilmette, IL (US); Ernest J. Boehm, Hanover Park, IL (US); Allen Erickson, Bixby, OK (US); Sudipta Ghosh, GURGAON (IN)

(21) Appl. No.: 18/455,294

(22) Filed: Aug. 24, 2023

(51) Int. Cl. C07C 7/04; C07C 7/00; C07C 7/09

(52) U.S. Cl. CPC C07C 7/04; C07C 7/005; C07C 7/09

(57) ABSTRACT

A process for separating an olefin stream from a methane stream is disclosed. The process comprises providing an olefin stream comprising C2 and/or C3 olefins. The olefin stream is cooled in a heat exchanger with a mixed refrigerant stream to provide a cooled olefin stream. The cooled olefin stream is passed to a demethanizer column operating at an overhead pressure of about 344 kPa gauge (50 psig) to about 2069 kPa gauge (300 psig). The cooled olefin stream is fractionated in the demethanizer column to provide a demethanizer column overhead vapor stream and a demethanizer column bottoms liquid stream. The process provides an improved recovery/yield of olefins and also optimizes the cooling and/or heating of other process streams.

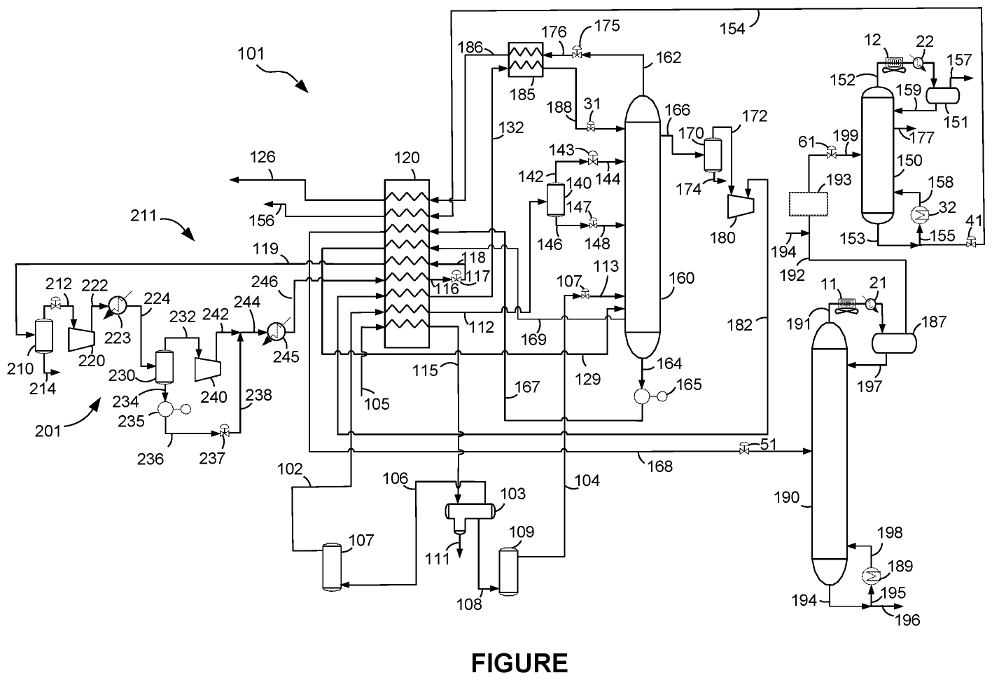

### FIELD

The field is related to a process for separating an olefin stream from a methane stream. The field may particularly relate to a process for cooling an olefin stream with a mixed refrigerant.

### BACKGROUND

There is an increasing support and demand across the globe for sustainable aviation fuels (SAF) with government offering subsidies and mandating the production of carbon-neutral jet fuel. In recent years, considerable research has been devoted in finding effective and efficient means of producing SAF. There are several different routes that can be taken to address the demand including the methanol-to-olefin route.

Olefins have been traditionally produced from petroleum feedstock by catalytic or steam cracking processes. These cracking processes, especially steam cracking, produce light olefins such as ethylene and propylene from a variety of hydrocarbon feedstocks. Ethylene and propylene are important commodity petrochemicals useful in a variety of processes for making plastics and other chemical compounds.

The petrochemical industry has known for some time that oxygenates, especially alcohols, are convertible into light olefins. For example, methanol, the preferred alcohol for light olefin production, may be converted to primarily ethylene and propylene in the presence of a molecular sieve catalyst. This process is referred to as a methanol-to-olefin (MTO) process, which occurs in an MTO reaction system. The highly efficient MTO process may convert oxygenates to light olefins which had been typically utilized in plastics production. Light olefins produced from the MTO process are concentrated in ethylene and propylene but includes C4-C6 olefins.

Usually, an ethylene rich stream is separated from light olefins by employing a process which recovers the ethylene component in a desirable, ethylene rich stream by separating it from other components and impurities. For example, depending on the feedstock composition, the reaction conditions, and the extent of side reactions, an MTO effluent can contain other light olefins and diolefins, and light paraffins such as methane and ethane. Some processes for separation involve the use of flash stages and distillation at cryogenic temperatures. While some processes for separation involve separating and recovering ethylene at non-cryogenic temperatures.

Cryogenic separation can be capital intensive due to both the capital cost of the specialized vessel metallurgy and refrigeration equipment, and the operating costs from compression and cooling. The compression and cooling may be provided by, for example, an ethylene refrigerant provided by an ethylene refrigeration compressor. On the other hand, the non-cryogenic temperatures may limit the extent of recovery of ethylene.

The methanol-to-jet process produces a large intermediate stream of light olefins at low pressure. A light olefin recovery process (LORP) compresses this stream and removes contaminants before light olefins can be oligomerized into jet fuel. Refrigeration and compression consume substantial energy in the LORP.

The need exists for improving recovery of olefins, reducing the equipment count, reducing the overall consumption of heat in the complex, and reducing overall emissions and operating costs.

### BRIEF SUMMARY

We have formulated a process and apparatus for separating an olefin stream from a methane stream using a mixed refrigerant system which provides an alternative to cascade refrigeration for light olefin recovery. The process involves the use of a mixed refrigerant which has several components of varying molecular weight that can be tailored to the specific process circumstances and fit a distinct boiling and cooling curve. Also, the mixed refrigerant system includes an integrated heat exchanger for heat exchanging other process streams with the mixed refrigerant stream.

### BRIEF DESCRIPTION OF THE DRAWINGS

The FIGURE a schematic drawing of a process for separating an olefin stream from a methane stream in accordance with an exemplary embodiment of the present disclosure.

### DEFINITIONS

The term “communication” means that fluid flow is operatively permitted between enumerated components, which may be characterized as “fluid communication”.

The term “downstream communication” means that at least a portion of fluid flowing to the subject in downstream communication may operatively flow from the object with which it fluidly communicates.

The term “upstream communication” means that at least a portion of the fluid flowing from the subject in upstream communication may operatively flow to the object with which it fluidly communicates.

The term “direct communication” or “directly” means that fluid flow from the upstream component enters the downstream component without passing through any other intervening vessel.

The term “column” means a distillation column or columns for separating one or more components of different volatilities. Unless otherwise indicated, each column includes a condenser on an overhead of the column to condense and reflux a portion of an overhead stream back to the top of the column and a reboiler at a bottom of the column to vaporize and send a portion of a bottoms stream back to the bottom of the column. Feeds to the columns may be preheated. The top pressure is the pressure of the overhead vapor at the vapor outlet of the column. The bottom temperature is the liquid bottom outlet temperature. Overhead lines and bottoms lines refer to the net lines from the column downstream of any reflux or reboil to the column. Stripping columns may omit a reboiler at a bottom of the column and instead provide heating requirements and separation impetus from a fluidized inert media such as steam. Stripping columns typically feed a top tray and take a product from the bottom.

As used herein, the term “separator” means a vessel which has an inlet and at least an overhead vapor outlet and a bottoms liquid outlet and may also have an aqueous stream outlet from a boot. A flash drum is a type of separator which may be in downstream communication with a separator that may be operated at higher pressure. As used herein, the term “boiling point temperature” means atmospheric equivalent boiling point (AEBP) as calculated from the observed boiling temperature and the distillation pressure, as calculated using the equations furnished in ASTM D1160 appendix A7 entitled “Practice for Converting Observed Vapor Temperatures to Atmospheric Equivalent Temperatures”.

As used herein, the term “True Boiling Point” (TBP) means a test method for determining the boiling point of a material which corresponds to ASTM D-2892 for the production of a liquefied gas, distillate fractions, and residuum of standardized quality on which analytical data can be obtained, and the determination of yields of the above fractions by both mass and volume from which a graph of temperature versus mass % distilled is produced using fifteen theoretical plates in a column with a 5:1 reflux ratio.

As used herein, the term “T5”, “T10”, “T90” or “T95” means the temperature at which 5 mass percent, 10 mass percent, 90 mass percent or 95 mass percent, as the case may be, respectively, of the sample boils using ASTM D-86 or TBP.

As used herein, the term “initial boiling point” (IBP) means the temperature at which the sample begins to boil using ASTM D-7169, ASTM D-86 or TBP, as the case may be.

As used herein, the term “end point” (EP) means the temperature at which the sample has all boiled off using ASTM D-7169, ASTM D-86 or TBP, as the case may be.

As used herein, the term “diesel” means hydrocarbons boiling in the range of an IBP between about 125° C. (257° F.) and about 175° C. (347° F.) or a T5 between about 150° C. (302° F.) and about 200° C. (392° F.) and the “diesel cut point” comprising a T95 between about 343° C. (650° F.) and about 399° C. (750° F.) using the TBP distillation method or a T90 between 280° C. (536° F.) and about 340° C. (644° F.) using ASTM D-86.

As used herein, the term “jet fuel” means hydrocarbons boiling in the range of a T10 between about 190° C. (374° F.) and about 215° C. (419° F.) and an end point (EP) of between about 290° C. (554° F.) and about 310° C. (590° F.).

As used herein, the term “a component-rich stream” means that the rich stream coming out of a vessel has a greater concentration of the component than the feed to the vessel and preferably than all other streams withdrawn from the vessel.

### DETAILED DESCRIPTION

For light olefin recovery, traditionally a cascade refrigeration is used. Cascade refrigeration usually involves typically four stages of propylene compression and between one and three stages of ethylene refrigeration. Typically, the cascade refrigeration is one of the largest utility consumers for light olefin recovery process and involves a lot of equipment for the individual chillers and the ancillary equipment in the refrigeration section like surge drums and pumps.

A process for separating an olefin stream from a methane stream is disclosed which provides an alternative to cascade refrigeration for light olefin recovery. The process involves the use of a mixed refrigerant, a refrigerant with several components that can be tailored to the specific process circumstances. The mixed refrigerant allows multiple exchangers to be combined into a single heat exchanger and involves fewer stages of compression. There is also a reduction in the power requirements under this arrangement. The mixed refrigerant system combines multiple heat exchangers according to their temperatures. For example, the mixed refrigerant system may be employed with low pressure demethanizer column, which typically uses a reflux compressor. The mixed refrigerant system may also be employed with a low pressure deethanizer column. In accordance with the present disclosure, a single refrigerant system is used to facilitate cooling and condensation for other process streams and at a wide temperature range. The disclosure employs a mixed refrigerant composition that provides sufficient heat exchange and/or cooling to the other process streams and at all of the temperature ranges exhibited in the process and apparatus.

A typical feed stream to the process includes an olefin stream comprising C2 and/or C3 olefins. Molecular sieves such as microporous crystalline zeolite and non-zeolitic catalysts, particularly silicoaluminophosphates (SAPO), are known to promote the conversion of oxygenates such as methanol to light olefins. The highly efficient methanol-to-olefin (MTO) process may convert oxygenates to light olefins which had been typically utilized for plastics production. Light olefins produced from the MTO process are concentrated in ethylene and propylene but include C4-C6 olefins. The olefin stream comprising C2 and/or C3 olefins may also comprise C4-C6 olefins.

The MTO process can produce an olefin stream comprising C2 and/or C3 olefins in varying ratios of C3:C2 depending on the operating conditions in the MTO reactor. An additional advantage of the mixed refrigerant arrangement is that the refrigerant composition can be fine-tuned during operations to optimize energy consumption. For example, an MTO process that is making olefins for oligomerization would operate at a higher C3:C2 ratio, which would correspond to a heavier mixed refrigerant.

Ethylene can be oligomerized into olefins such as C4, C6 and C8 olefins. Propylene can be oligomerized into olefins such as C6, C9 and C12 olefins. Larger MTO olefins can also be oligomerized. Olefin oligomerization is a process that can oligomerize smaller olefins into larger olefins. More specifically, olefin oligomerization can convert olefins into distillates including jet fuel and diesel range products. The oligomerized distillate can be saturated for use as transportation fuels.

In an exemplary embodiment, the olefin stream comprising C2 and/or C3 olefins is produced from reacting oxygenates over a SAPO catalyst in a MTO process.

In an exemplary embodiment, the MTO process and the demethanizer of the present disclosure are part of a renewable fuels and/or renewable olefins complex wherein the oxygenate feed to the MTO process is derived from renewable sources. The operating conditions in the demethanizer and the mixed refrigerant will be dependent on the mix of renewable products desired from the unit and would be tailored to the specific needs of the process.

Turning to the FIGURE showing a process 101 for separating an olefin stream from a methane stream comprising a demethanizer column 160, a deethanizer column 190, a C2 splitter column 150, and a mixed refrigerant system 201. In accordance with the present disclosure, the mixed refrigerant system 201 comprises a mixed refrigerant loop 211 and a cryogenic heat exchanger 120. The methane stream, in accordance with the present disclosure, may comprise other light components along with the methane. The methane stream may comprise nitrogen (N₂), carbon monoxide (CO), hydrogen (H₂), and methane (CH₄).

In an exemplary embodiment, a wet olefin feed stream comprising C2 and/or C3 olefins in line 105 is delivered to the process 101. In a typical embodiment, the wet olefin feed stream may be compressed to a pressure of about 1378 kPa gauge (200 psig) to about 2414 kPa gauge (350 psig) and scrubbed with caustic and water to eliminate most oxygenates and carbon dioxide. The wet olefin feed stream 105 may be passed to the demethanizer column 160.

In an aspect, the wet olefin feed stream in line 105 may be passed to the cryogenic heat exchanger 120. The wet olefin feed stream in line 105 may be passed to the cryogenic heat exchanger 120 at a temperature of about 37° C. (100° F.) to about 66° C. (150° F.). In an embodiment, the wet olefin feed stream in line 105 includes a light olefinic vapor stream from the MTO process. In an exemplary embodiment, the light olefinic vapor stream from the MTO process may comprise C2 and/or C3 olefins. In another exemplary embodiment, the wet olefin feed stream in line 105 may include a scrubbed light olefinic vapor stream from the MTO process.

The wet olefin feed stream in line 105 may be cooled by heat exchange in the heat exchanger 120 to liquefy a part of the stream and provide a cooled wet olefin feed stream in line 115. In an exemplary embodiment, the cooled wet olefin feed stream in line 115 is at a temperature of about 5° C. (40° F.) to about 32° C. (90° F.). The cooled wet olefin feed stream in line 115 may be separated in a separator 103 to provide an aqueous stream from a boot in line 111 and a wet olefin vapor feed stream comprising C2− hydrocarbons and gases in an overhead line 106 and a wet olefin liquid feed stream in a bottoms line 108 comprising C3+ hydrocarbons. The wet olefin vapor feed stream in the overhead line 106 is dried in a drier 107 to provide the first olefin vapor feed stream in line 102. The wet olefin liquid feed stream in the bottoms line 108 is dried in a drier 109 to provide the first olefin liquid feed stream in line 104. The first olefin vapor feed stream in line 102 and the first olefin liquid feed stream in line 104 are sent to demethanizer column 160 as described herein below.

In an aspect, the wet olefin feed stream comprising C2 and/or C3 olefins in line 105 is separated into a first olefin vapor feed stream in line 102 and a first olefin liquid feed stream in line 104 and enter the process as such. The first olefin vapor feed stream in line 102 and the first olefin liquid feed stream in line 104 may be passed to the demethanizer column 160 separately. In an aspect, the first olefin vapor feed stream in line 102 is passed to the cryogenic heat exchanger 120 at a pressure of about 1380 kPa gauge (200 psig) to about 2414 kPa gauge (350 psig). The first olefin vapor feed stream in line 102 is cooled in the cryogenic heat exchanger 120 via heat exchange in the cryogenic heat exchanger 120 to provide a cooled first olefin vapor feed stream in line 112. In an exemplary embodiment, the cooled first olefin vapor feed stream in line 112 is at a temperature of about −56° C. (−70° F.) to about −90° C. (−130° F.). The cooled first olefin vapor feed stream in line 112 is passed to the demethanizer column 160.

In accordance with an exemplary embodiment of the present disclosure, the cooled first olefin vapor feed stream in line 112 may be separated before passing it to the demethanizer column 160. The cooled first olefin vapor feed stream in line 112 may be passed to a separator 140 to provide a second olefin vapor feed stream in line 142 and a second olefin liquid feed stream in line 146. The second olefin vapor feed stream in line 142 and the second olefin liquid feed stream in line 146 are passed to the demethanizer column 160. The second olefin vapor feed stream in line 142 may be expanded by an expansion valve 143 to provide an expanded second olefin vapor feed stream in line 144. In an exemplary embodiment, the expanded second olefin vapor feed stream in line 144 is at a temperature of about −62° C. (−80° F.) to about −102° C. (−150° F.) The expanded second olefin vapor feed stream in line 144 is passed to the demethanizer column 160. Alternatively, the second olefin vapor feed stream in line 142 may be passed directly to the demethanizer column 160.

The second olefin liquid feed stream in line 146 can be expanded by an expansion valve 147 to provide an expanded second olefin liquid feed stream in line 148. In an exemplary embodiment, the expanded second olefin liquid feed stream in line 148 is at a temperature of about −51° C. (−60° F.) to about −90° C. (−130° F.). The expanded second olefin liquid feed stream in line 148 is passed to the demethanizer column 160. Alternatively, the second olefin liquid feed stream in line 146 may be passed directly to the demethanizer column 160. In an aspect of the present disclosure, the use of separator 140 for separating the cooled first olefin vapor feed stream in line 112 is optional. Thus, the cooled first olefin vapor feed stream in line 112 may be passed directly to the demethanizer column 160 and separated therein.

The first olefin liquid feed stream in line 104 may be passed to the demethanizer column 160. The first olefin liquid feed stream in line 104 can be expanded by an expansion valve 107 to provide an expanded first olefin liquid feed stream in line 113. The expanded first olefin liquid feed stream in line 113 is passed to the demethanizer column 160.

In the demethanizer column 160, the expanded second olefin vapor feed stream in line 144, the expanded second olefin liquid feed stream in line 148 and the expanded liquid olefin stream in line 113 are fractionated to provide a demethanizer column overhead vapor stream comprising methane and lighter gases in an overhead line 162 and a demethanizer column bottoms liquid stream comprising C2 and larger hydrocarbons in a bottoms line 164. In accordance with the present disclosure, the demethanizer column 160 operates at an overhead pressure of about 344 kPa gauge (50 psig) to about 2069 kPa gauge (300 psig). In an exemplary embodiment, the demethanizer column overhead vapor stream in the overhead line 162 is at a temperature of about −101° C. (−150° F.) to about −135° C. (−210° F.). In another exemplary embodiment, the demethanizer column overhead vapor stream in line 162 is at a temperature of about −112° C. (−170° F.) to about −129° C. (−200° F.).

The traditionally used cascade refrigeration process employs two separate refrigeration loops. The first loop usually has two to four stages of typically propylene compression and second loop usually has between one and three stages of ethylene refrigeration to cool a light olefin feed stream sufficiently to separate methane from heavier hydrocarbons. The process in accordance with the present disclosure employs a mixed refrigerant system 201 which allows the demethanizer column 160 to operate at a lower overhead pressure of about 344 kPa gauge (50 psig) to about 2069 kPa gauge (300 psig) as compared to a higher overhead pressure of about 2896 kPa gauge (420 psig) in a cascade refrigerated demethanizer column. However, the demethanizer column 160 may be run at a higher pressure of about 2069 kPa gauge (300 psig) to about 3964 kPa gauge (575 psig) under certain circumstances if advantageous. In the current process of processing the olefin stream, the demethanizer column 160 can be operated at a lower pressure of about 517 kPa gauge (75 psig) to about 1379 kPa (200 psig) or a lower pressure of about 689 kPa gauge (100 psig) to about 1035 kPa (150 psig). Also, the process with the mixed refrigerant system 201 omits the fourth stage of compression for the wet olefin feed stream in line 105 or the first olefin vapor feed stream in line 102, typically used with the cascade refrigerated demethanizer column since the demethanizer column 160 herein can be operated at lower pressure. Furthermore, the demethanizer column 160 cooled with a mixed refrigerant system 201 provides an additional recovery of ethylene of about 0.5 to about 1.5 wt % as compared to the process with a cascade refrigerated demethanizer column. Operating the demethanizer column 160 at a lower overhead pressure has several advantages and benefits including a lower capital expense, easier separation, lesser duty or energy requirement, and fewer fractionation stages for separation in the column.

The overhead, the bottom and side of the demethanizer column 160 are all in fluid and heat communication with the cryogenic heat exchanger 120 of the mixed refrigerant system 201.

In an aspect of the present disclosure, a first side stream is withdrawn in line 166 from a side of the demethanizer column 160. In an exemplary embodiment, the first side stream is a vapor side stream in line 166. In an aspect, the vapor side stream is withdrawn from a location above a location of an inlet for an expanded second olefin vapor feed stream in line 144 and below a location for an inlet for a demethanizer column reflux stream in line 188 to the demethanizer column 160. The first side stream is compressed, chilled, and returned to the overhead of the demethanizer column 160 to provide reflux for the column to meet the required ethylene recovery. As shown in the FIGURE, a first side stream in line 166 is separated by the demethanizer column 160. The first side stream in line 166 may be passed to the cryogenic heat exchanger 120 to exchange heat with the mixed refrigerant stream in line 246 and with the other process streams. In an aspect, the first side stream in line 166 may be separated in an overhead knock out drum (KOD) 170 before it is passed to the cryogenic heat exchanger 120. The first side stream in line 166 is separated in the overhead KOD 170 into a KOD overhead vapor stream in line 172 and a knocked out liquid stream in line 174. From the overhead KOD 170, the KOD overhead vapor stream in line 172 is withdrawn and passed to a reflux compressor 180 to provide a compressed first side stream in line 182. The KOD 170 may be used to separate any liquid present in the first side stream in line 166 before passing the first side stream to the downstream reflux compressor 180. In an aspect, the first side stream in line 166 may be passed directly to the reflux compressor 180 to provide the compressed first side stream in line 182.

In an exemplary embodiment, the KOD overhead vapor stream in line 172 is compressed to a pressure of about 2757 kPa gauge (400 psig) to about 3793 kPa gauge (550 psig) in the reflux compressor 180 to provide the compressed first side stream in line 182. In an embodiment, the compressed first side stream in line 182 is at a temperature of about 2° C. (35° F.) to about 32° C. (90° F.). The compressed first side stream in line 182 is passed to the cryogenic heat exchanger 120 to provide a demethanizer column reflux stream in line 132. The compressed first side stream in line 182 is cooled by heat exchange in the cryogenic heat exchanger 120 to provide the demethanizer column reflux stream in line 132. In an embodiment, the demethanizer column reflux stream in line 132 is at a temperature of about −73° C. (−100° F.) to about −129° C. (−200° F.). In an aspect, a portion of the compressed first side stream in line 182 may be recycled to the KOD 170 and the other portion may be cooled by heat exchange in the cryogenic heat exchanger 120 to provide the demethanizer column reflux stream in line 132, but this aspect is not shown.

The demethanizer column overhead vapor stream in line 162 is withdrawn and passed to the cryogenic heat exchanger 120 for heat exchange with the mixed refrigerant and other process streams. In an embodiment, the demethanizer column overhead vapor stream in line 162 is passed through an overhead heat exchanger 185 before it is passed to the cryogenic heat exchanger 120. In the overhead heat exchanger 185 the demethanizer column overhead vapor stream in line 162 may be warmed by another process stream of the demethanizer column 160. In an embodiment, the demethanizer column overhead vapor stream in line 162 may be warmed by a first side stream in line 132 of the demethanizer column 160 and the first side stream in line 132 may be cooled by the demethanizer column overhead vapor stream in line 162. In an aspect, the demethanizer column overhead vapor stream in line 162 may be expanded via an expansion valve 175 to provide an expanded demethanizer column overhead vapor stream in line 176 which is passed to the overhead heat exchanger 185.

Alternatively, the demethanizer column overhead vapor stream in line 162 may be expanded in an expander (not shown) for some extra cooling and perhaps power recovery to provide an expanded demethanizer column overhead vapor stream in line 176. Expansion may reduce the temperature by about 3° C. (5° F.) to about 8° C. (15° F.). Further, the demethanizer column overhead vapor stream in line 162 may be passed directly to the overhead heat exchanger 185. In an exemplary embodiment, the expanded demethanizer column overhead vapor stream in line 176 is at a temperature of about −112° C. (−170° F.) to about −140° C. (−220° F.). From the overhead heat exchanger 185, a heated demethanizer column overhead vapor stream in line 186 is passed to the cryogenic heat exchanger 120. In an aspect, the expanded demethanizer column overhead vapor stream in line 176 may be condensed by heat exchange in the overhead heat exchanger 185 with the demethanizer column reflux stream in line 132 from the demethanizer column 160. In an exemplary embodiment, the first heated demethanizer column overhead vapor stream in line 186 is at a temperature of about 15° C. (60° F.) to about 32° C. (90° F.).

In the overhead heat exchanger 185, the demethanizer column reflux stream in line 132 is further cooled after heat exchange with the expanded demethanizer column overhead vapor stream in line 176 to provide a heat exchanged demethanizer column reflux stream in line 188. In an exemplary embodiment, the heat exchanged demethanizer column reflux stream in line 188 is at a temperature of about −101° C. (−150° F.) to about −124° C. (−190° F.). The heat exchanged demethanizer column reflux stream in line 188 is passed to the demethanizer column 160 near the overhead of the demethanizer column 160. In an exemplary embodiment, the heat exchanged demethanizer column reflux stream in line 188 may be passed the demethanizer column 160 through an expansion valve 31.

In an embodiment, the cryogenic heat exchanger 120 and overhead heat exchanger 185 are separate heat exchangers. These heat exchangers may be plate and fin type (also called brazed aluminum type), printed circuit type, plate and frame type, or shell and tube type heat exchangers. In an alternative embodiment, cryogenic heat exchanger 120 and overhead heat exchanger 185 may be combined into a single heat exchanger 120. In a further alternative embodiment, cryogenic heat exchanger 120 may be separated into two or more heat exchangers. The decision to combine or separate heat exchangers depends on the thermal characteristics of the constituent streams, the properties of the heat exchanger technology selected, and the cost of construction of the heat exchangers.

A second side stream in line 169 is also withdrawn from the demethanizer column 160. In an aspect, the second side stream in line 169 is a liquid stream. In an exemplary embodiment, the second side stream in line 169 is a reboiler stream of the demethanizer column 160. The second side stream in line 169 may be reboiled in the cryogenic heat exchanger 120. In an exemplary embodiment, the second side stream in line 169 is at a temperature of about −32° C. (−26° F.) to about 10° C. (50° F.). The second side stream in line 169 is passed to the cryogenic heat exchanger 120 where it is reboiled. The second side stream in line 169 is heated by heat exchange in the cryogenic heat exchanger 120 to provide a heated second side stream in line 129. The heated second side stream in line 129 is withdrawn from the cryogenic heat exchanger 120. In an embodiment, the heated second side stream in line 129 is at a temperature of about −23° C. (−10° F.) to about 16° C. (60° F.). The heated second side stream in line 129 may be passed to the demethanizer column 160 at a location above the location from which the second side stream is taken in line 169 from the demethanizer column 160. Alternatively, the heated second side stream in line 129 may be passed to the demethanizer column 160 at a location below the location from which the second side stream is taken in line 169. Although not shown, another reboiler stream may be withdrawn from the demethanizer column 160. The another reboiler stream may be withdrawn from a suitable location from above or below the location at which the second side stream is taken in line 169 from the demethanizer column 160. The another reboiler stream may be heat exchanged in the cryogenic heat exchanger 120 and recycled back to the demethanizer column 160.

From the bottom of the demethanizer column 160, a demethanizer column bottoms liquid stream in line 164 is withdrawn. In an exemplary embodiment, the demethanizer column bottoms liquid stream in line 164 is at a temperature of about −20° C. (−5° F.) to about −45° C. (−50° F.). In the cryogenic heat exchanger 120, the demethanizer column bottoms liquid stream in line 164 is heat exchanged with the other process streams and with the mixed refrigerant stream in line 246. The demethanizer column bottoms liquid stream in line 164 may be sent to a pump 165 and a pumped demethanizer column bottoms liquid stream in line 167 is passed to the cryogenic heat exchanger 120.

In the cryogenic heat exchanger 120, the pumped demethanizer column bottoms liquid stream in line 167 is heat exchanged with the mixed refrigerant stream in line 246 and with the other process streams. In an embodiment, the pumped demethanizer column bottoms liquid stream in line 167 is heated by heat exchange in the cryogenic heat exchanger 120 to provide a heated demethanizer column bottoms liquid stream in line 168. The heated demethanizer column bottoms liquid stream in line 168 is withdrawn from the cryogenic heat exchanger 120. In an embodiment, the heated demethanizer column bottoms liquid stream in line 168 is at a temperature of about 5° C. (40° F.) to about 38° C. (100° F.). The heated demethanizer column bottoms liquid stream in line 168 may be separated and used for further processing. In an aspect of the present disclosure, the heated demethanizer column bottoms liquid stream in line 168 may passed as a feed to the deethanizer column 190. In an aspect, the heated demethanizer column bottoms liquid stream in line 168 may be passed to the deethanizer column 190 through an expansion valve 51. In accordance with an embodiment of the present disclosure, the deethanizer column 190 is in downstream communication with the demethanizer column 160. In accordance with another embodiment of the present disclosure, the deethanizer column 190 is in upstream communication with the demethanizer column 160.

In an aspect of the present disclosure, the mixed refrigerant system 201 is used to heat exchange with other process streams in the cryogenic heat exchanger 120 with the mixed refrigerant stream in line 246. In an exemplary embodiment, the other process streams that may be heat exchanged in the cryogenic heat exchanger 120 include the first olefin vapor feed in line 102, the demethanizer column overhead vapor stream in line 162, a first side stream in line 166, the second side stream in line 169, the demethanizer column bottoms liquid stream in line 164 and the wet olefin feed stream in line 105. Optionally a net C2 splitter bottoms liquid stream in line 154 may be passed to heat exchanger and heated with the other process streams.

In the cryogenic heat exchanger 120, the first heated demethanizer overhead vapor stream in line 186 is heat exchanged with the mixed refrigerant stream in line 246 and with the other streams to provide a second heated demethanizer overhead vapor stream in line 126. The first heated demethanizer overhead vapor stream in line 186 is warmed after heat exchange in the cryogenic heat exchanger 120 to provide the second heated demethanizer overhead vapor stream in line 126. The second heated demethanizer overhead vapor stream in line 126 is withdrawn from the cryogenic heat exchanger 120. In an embodiment, the second heated demethanizer overhead vapor stream in line 126 is at a temperature of about 10° C. (50° F.) to about 38° C. (100° F.). The second heated demethanizer overhead vapor stream in line 126 may be separated and passed to a fuel gas header. The demethanizer overhead vapor stream in line 126 may also be used for reactor purges in the MTO unit, fuel for the CO boiler in the MTO unit, and/or fuel for the reboiler heater in an oligomerization unit.

Returning to the mixed refrigerant system 201 in the FIGURE, the mixed refrigerant system 201 is provided for heat exchanging the mixed refrigerant with the other process streams as described herein above in the cryogenic heat exchanger 120. The mixed refrigerant system 201 operates with a refrigerant stream that may comprise a mixed refrigerant stream comprising an inert gas and some or all of C1 to C5 hydrocarbons. In an exemplary embodiment, the mixed refrigerant composition may comprise about 0 mol % to about 25 mol % C1 hydrocarbon, about 25 mol % to about 40 mol % C2 hydrocarbon, and about 20 mol % to about 50 mol % C3 hydrocarbon. The inert gas may include nitrogen in an amount from about 0 mol % to about 20 mol %. The C2 hydrocarbon may be ethane or ethylene, and the C3 hydrocarbon may be propane or propylene. In an exemplary embodiment, the mixed refrigerant system 201 may be operated with a refrigerant stream comprising non-hydrocarbon refrigerants. Other suitable refrigerant components may be used in the mixed refrigerant system 201.

The mixed refrigerant system 201 includes a mixed refrigerant loop 211 and the cryogenic heat exchanger 120. As shown, a mixed refrigerant stream in line 246 of the mixed refrigerant system 201 is passed to the cryogenic heat exchanger 120. In an embodiment, a mixed refrigerant stream in line 246 is passed to the cryogenic heat exchanger 120 after compression to higher pressure. In an exemplary embodiment, the mixed refrigerant stream in line 246 has a pressure of about 689 kPa gauge (100 psig) to about 2414 kPa gauge (350 psig).

After heat exchange, a cooled mixed refrigerant stream in line 116 is withdrawn from the cryogenic heat exchanger 120. In an exemplary embodiment, the cooled mixed refrigerant stream in line 116 may be withdrawn at a temperature of about −74° C. (−101° F.) to about −129° C. (−200° F.). The cooled mixed refrigerant stream in line 116 may be passed through an expansion valve 117 to provide an expanded mixed refrigerant stream in line 118. In an exemplary embodiment, the expanded mixed refrigerant stream in line 118 is expanded to a pressure of about 137 kPa gauge (20 psig) to about 414 kPa gauge (60 psig) via the expansion valve 117. In another exemplary embodiment, the expanded mixed refrigerant stream in line 118 is at a temperature of about −84° C. (−120° F.) to about −129° C. (−200° F.). The expanded mixed refrigerant stream in line 118 is returned to the cryogenic heat exchanger 120 for further heat exchange. In an aspect, the expanded mixed refrigerant stream in line 118 is warmed via heat exchange in the cryogenic heat exchanger 120 to a temperature of about 21° C. (70° F.) to about 93° C. (200° F.).

After further heat exchange in the cryogenic heat exchanger 120, a warm heat exchanged mixed refrigerant stream in line 119 is withdrawn from the cryogenic heat exchanger 120. The warm heat exchanged mixed refrigerant stream in line 119 is processed in the mixed refrigerant loop 211 of the mixed refrigerant system 201 to provide the mixed refrigerant stream 246.

In an embodiment of the present disclosure, the warm heat exchanged mixed refrigerant stream in line 119 is compressed in a multistage compressor of the refrigerant loop 211. In an exemplary embodiment, the multistage compressor is a two-stage refrigerant compressor comprising a first stage refrigerant compressor 220 and a second stage refrigerant compressor 240.

In accordance with the present disclosure, the warm heat exchanged mixed refrigerant stream in line 119 is passed to a first suction drum 210 to provide a first overhead mixed refrigerant stream in line 212 and a first bottoms mixed refrigerant stream in line 214. The first overhead mixed refrigerant stream in line 212 of the first suction drum 210 is compressed in the first stage refrigerant compressor 220 to provide a first compressed mixed refrigerant stream in line 222. In an exemplary embodiment, the first overhead mixed refrigerant stream in line 212 is at a pressure of about 40 kPa (g) (10 psig) to about 344 kPa (g) (50 psig). In an aspect, the first overhead mixed refrigerant stream in line 212 may be compressed in the first stage refrigerant compressor 220 to provide the first compressed mixed refrigerant stream in line 222. In an exemplary embodiment, the first compressed mixed refrigerant stream in line 222 is at a pressure of about 344 kPa gauge (50 psig) to about 1379 kPa gauge (200 psig). In an exemplary embodiment, the first compressed mixed refrigerant stream in line 222 is at a temperature of about 65° C. (150° F.) to about 121° C. (250° F.).

The first compressed mixed refrigerant stream in line 222 may be cooled in a cooler 223 to provide a first cooled compressed mixed refrigerant stream in line 224. The cooler 223 may be an air cooler or a water cooler. Alternatively, any suitable stream from the process may be used to cool the first compressed mixed refrigerant stream in line 222. In an exemplary embodiment, the first compressed mixed refrigerant stream in line 222 may be cooled to a temperature of about 21° C. (70° F.) to about 65° C. (150° F.) in the cooler 223.

The first cooled compressed mixed refrigerant stream in line 224 is passed to a second suction drum 230 to provide a second overhead mixed refrigerant stream in line 232 and a second bottoms mixed refrigerant stream in line 234. The second overhead mixed refrigerant stream in line 232 is compressed in the second stage refrigerant compressor 240 to provide a second compressed mixed refrigerant stream in line 242. In an aspect, the second overhead mixed refrigerant stream in line 232 is compressed to a pressure of about 1724 kPa gauge (250 psig) to about 2758 kPa gauge (400 psig) in the second stage refrigerant compressor 240. In an exemplary embodiment, the second compressed mixed refrigerant stream in line 242 is at a temperature of about 65° C. (150° F.) to about 121° C. (250° F.).

The second bottoms mixed refrigerant stream in line 234 is pumped through a pump 235. A pumped second bottoms mixed refrigerant stream in line 236 is passed through an expansion valve 237 to provide an expanded second bottoms mixed refrigerant stream in line 238. In an exemplary embodiment, pumped second bottoms mixed refrigerant stream in line 236 is at a temperature of about 26° C. (80° F.) to about 65° C. (150° F.).

In an aspect, the second compressed mixed refrigerant stream in line 242 and the expanded second bottoms mixed refrigerant stream in line 238 may be combined to provide a compressed mixed refrigerant stream in line 244. In an exemplary embodiment of the present disclosure, the compressed mixed refrigerant stream in line 244 is at a temperature of about 48° C. (120° F.) to about 104° C. (220° F.). In an embodiment, the compressed mixed refrigerant stream in line 244 may be passed through a cooler 245 to provide a mixed refrigerant stream in line 246 having a temperature of about 26° C. (80° F.) to about 40° C. (104° F.). The mixed refrigerant stream in line 246 is passed to the cryogenic heat exchanger 120. The cooler 245 may be an air cooler or a water cooler. Alternatively, any suitable stream from the process may be used to cool the compressed mixed refrigerant stream in line 244. It is also contemplated that the first bottoms mixed refrigerant stream in line 214 can be combined with the second bottoms mixed refrigerant stream in line 234 and pumped and processed therewith.

Referring to the deethanizer column 190, the heated demethanizer bottoms liquid stream in line 168 is fractionated in the deethanizer column 190. The deethanizer column 190 fractionates the heated demethanizer bottoms liquid stream in line 168 into a deethanizer overhead vapor stream in line 191 rich in C2 hydrocarbons and a total deethanizer bottoms liquid stream in line 194 rich in C3+ hydrocarbons. The deethanizer overhead vapor stream in line 191 may be passed to a C2 splitter column 150. The deethanizer overhead vapor stream in line 191 may be separated into liquid and vapor streams before passing it to the C2 splitter column 150. In an aspect, the deethanizer overhead vapor stream in line 191, after passing through a deethanizer condenser 21, may be fed to a deethanizer receiver 187 in which it is separated into a deethanizer reflux stream in line 197 and a deethanizer receiver overhead vapor stream in line 192. Optionally, there may be a deethanizer cooler 11 for cooling the deethanizer overhead vapor stream in line 191 before passing it to the deethanizer condenser 21. The deethanizer condenser 21 may be cooled with a propylene refrigerant stream. Alternatively, the deethanizer condenser 21 may be cooled with the mixed refrigerant stream in line 246, the deethanizer condenser 21 may be combined with the cryogenic heat exchanger 120, or the deethanizer condenser 21 may be combined with a C2 splitter reboiler exchanger 32. The deethanizer reflux stream in line 197 from the deethanizer receiver 187 may be returned back to the deethanizer column 190. In an embodiment, the deethanizer receiver overhead vapor stream in line 192 is fed to the C2 splitter column 150. In an alternate embodiment, the deethanizer receiver overhead vapor stream in line 192 may be fed to an oligomerization unit (not shown).

The total deethanizer bottoms liquid stream in line 194 provides a deethanizer reboiler liquid stream in line 195 which is heated by heat exchange in a deethanizer bottoms reboiler exchanger 189. A heated deethanizer reboiler liquid stream in line 198 is fed back to the deethanizer column 190. The total deethanizer bottoms liquid stream in line 194 also provides a net deethanizer bottoms liquid stream in line 196. In an exemplary embodiment, the net deethanizer bottoms liquid stream in line 196 may be fed to a depropanizer column (not shown). The deethanizer column 190 may be operated at an overhead temperature of about −4° C. (25° F.) to about −37° C. (−35° F.) and a bottoms gauge pressure of about 1.7 MPa (g) (250 psig) to about 2.9 MPa (g) (425 psig).

In an exemplary embodiment, the deethanizer receiver overhead vapor stream in line 192 may be combined with a hydrogen stream in line 194 and passed to an acetylene removal unit 193 to convert acetylene present in the stream in line 192 to ethylene by selective hydrogenation. A broad range of suitable operating pressures in the acetylene removal unit 193 range from about 276 kPag (40 psig) to about 5516 kPag (800 psig), or about 345 kPag (50 psig) to about 2069 kPag (300 psig). A relatively moderate temperature between about 25° C. (77° F.) and about 350° C. (662° F.), or about 50° C. (122° F.) to about 200° C. (392° F.) is typically employed. The vapor hourly space velocity of the reactants for the selective hydrogenation catalyst may be about 30 hr−1, or above about 300 hr−1, or above about 15 hr−1, to about 600 hr−1. To avoid the undesired saturation of a significant amount mono-olefinic hydrocarbons, the mole ratio of hydrogen to multi-olefinic hydrocarbons in the material entering the bed of selective hydrogenation catalyst is maintained between 0.75:1 and 1.8:1.

A selective hydrogenation catalyst may be any suitable catalyst which is capable of selectively hydrogenating acetylene in a C2− hydrocarbon stream. A particularly preferred selective hydrogenation catalyst comprises copper and at least one other metal such as titanium, vanadium, chrome, manganese, cobalt, nickel, zinc, molybdenum, and cadmium or mixtures thereof. The metals are preferably supported on inorganic oxide supports such as silica and alumina. Preferably, a selective hydrogenation catalyst may comprise a copper and a nickel metal supported on alumina. The selectively hydrogenated effluent may be dried to remove moisture. From the acetylene removal unit 193, an acetylene-free deethanizer receiver overhead vapor stream is withdrawn. In an aspect, the acetylene-free deethanizer receiver overhead vapor stream is expanded through an expansion valve 61. From the expansion valve 61, an expanded deethanizer receiver overhead vapor stream in line 199 may be passed to the C2 splitter column 150.

The C2 splitter column 150 fractionates the expanded deethanizer receiver overhead vapor stream in line 199 from the deethanizer column 190 into a first C2 splitter overhead vapor stream comprising hydrogen and light ends in line 152, a C2 splitter sidedraw stream rich in ethylene in line 177, and a total C2 splitter bottoms liquid stream in line 153 rich in ethane. In an aspect, the C2 splitter overhead vapor stream in line 152, after passing through a C2 splitter condenser 22, may be fed to a C2 splitter receiver 151 in which it is separated into a C2 splitter reflux stream in line 159 and a C2 splitter receiver overhead vapor stream in line 157. Optionally, there may be a C2 splitter cooler 12 for cooling the C2 splitter overhead vapor stream in line 152 before passing it to the C2 splitter condenser 22. The C2 splitter condenser 22 may be cooled with a propylene refrigerant stream. Alternatively, the C2 splitter condenser 22 may be cooled with the mixed refrigerant stream in line 246 or the C2 splitter condenser 22 may be combined with the cryogenic heat exchanger 120. A C2 splitter reflux stream in line 159 from the C2 splitter receiver 151 is returned back to the C2 splitter column 150. The C2 splitter sidedraw stream in line 177 is the ethylene product stream that is sent to storage or downstream processing. The C2 splitter receiver overhead vapor stream in line 157 may be sent to the fuel gas header or returned to the upstream MTO unit as a reactor purge.

The total C2 splitter bottoms liquid stream in line 153 provides a C2 splitter reboiler liquid stream in line 155 which is heated by heat exchange in a C2 splitter bottoms reboiler exchanger 32. A heated C2 splitter reboiler liquid stream in line 158 is fed back to the C2 splitter column 150. The total C2 splitter bottoms liquid stream in line 153 also provides a net C2 splitter bottoms liquid stream in line 154. The C2 splitter column 190 may be operated at a bottoms temperature of about −17° C. (0° F.) to about 5° C. (40° F.) and an overhead gauge pressure of about 1 MPa (g) (150 psig) to about 2.4 MPa (g) (350 psig).

Optionally, the net C2 splitter bottoms liquid stream in line 154 may be passed to the cryogenic heat exchanger 120. In an aspect, the net C2 splitter bottoms liquid stream in line 154 may be passed to the cryogenic heat exchanger 120 through an expansion valve 41. In the cryogenic heat exchange 120, the net C2 splitter bottoms liquid stream in line 154 is heated after heat exchange in the cryogenic heat exchanger 120. In an exemplary embodiment, the net C2 splitter bottoms liquid stream in line 154 may be passed to the cryogenic heat exchanger 120 at a temperature of about −23° C. (−10° F.) to about −68° C. (−90° F.). A heated net C2 splitter bottoms stream in line 156 is withdrawn from the cryogenic heat exchanger 120. In an exemplary embodiment, the heated net C2 splitter bottoms stream in line 156 is at a temperature of about 5° C. (40° F.) to about 38° C. (100° F.). The heated net C2 splitter bottoms stream in line 156 may be separated and passed to a fuel gas header or to further processing or upgrading.

There are several heat exchangers in the disclosure that can be integrated with other process streams in nearby services. In an exemplary embodiment, the deethanizer bottoms reboiler exchanger 189 may exchange heat with hot water streams from the MTO process. In another exemplary embodiment, the deethanizer bottoms reboiler exchanger 189 may exchange heat with an oligomerization reactor effluent and/or hydrogenation reactor effluent in an oligomerization unit. In yet another exemplary embodiment, the first stage refrigeration condenser 223 and/or second stage refrigeration condenser 245 may exchange heat with the deethanizer bottoms reboiler exchanger 189 and/or C2 splitter bottoms reboiler exchanger 32.

### Specific Embodiments

While the following is described in conjunction with specific embodiments, it will be understood that this description is intended to illustrate and not limit the scope of the preceding description and the appended claims.

A first embodiment of the present disclosure is a process for separating an olefin stream from a methane stream comprising providing an olefin stream comprising C2 and/or C3 olefins; cooling the olefin stream in a heat exchanger with a mixed refrigerant stream to provide a cooled olefin stream; passing the cooled olefin stream to a demethanizer column operating at an overhead pressure of about 344 kPa gauge (50 psig) to about 2069 kPa gauge (300 psig); and fractionating the cooled olefin stream in the demethanizer column to provide a demethanizer column overhead vapor stream and a demethanizer column bottoms liquid stream. An embodiment of the present disclosure is one, any or all of prior embodiments in this paragraph up through the first embodiment in this paragraph wherein the olefin stream is separated into a vapor olefin stream and a liquid olefin stream which are passed to the demethanizer column separately. An embodiment of the present disclosure is one, any or all of prior embodiments in this paragraph up through the first embodiment in this paragraph further comprises passing the vapor olefin stream to the heat exchanger; cooling the vapor olefin stream in the heat exchanger to provide a cooled vapor olefin stream; separating the cooled vapor olefin stream to provide an overhead vapor olefin stream and a bottom liquid olefin stream; and fractionating the overhead vapor olefin, the bottom liquid olefin stream, and the liquid olefin stream in the demethanizer column. An embodiment of the present disclosure is one, any or all of prior embodiments in this paragraph up through the first embodiment in this paragraph further comprising taking a first side stream from the demethanizer column; passing the first side stream to the heat exchanger to provide a heat exchanged first side stream; and passing the heat exchanged first side stream to the demethanizer column. an embodiment of the present disclosure is one, any or all of prior embodiments in this paragraph up through the first embodiment in this paragraph further comprising passing the first side stream to a reflux compressor to provide a compressed first side stream; passing the compressed first side stream to the heat exchanger to provide a heat exchanged first side stream; passing the heat exchanged first side stream to the demethanizer column. an embodiment of the present disclosure is one, any or all of prior embodiments in this paragraph up through the first embodiment in this paragraph further comprising passing the heat exchanged first side stream to an overhead heat exchanger to provide a cooled reflux stream; expanding the cooled reflux stream to provide an expanded reflux stream; and passing the expanded reflux stream to the demethanizer column. an embodiment of the present disclosure is one, any or all of prior embodiments in this paragraph up through the first embodiment in this paragraph further comprising taking a second side stream from the demethanizer column; passing the second side stream to the heat exchanger to provide a heated second side stream; and passing the heated second side stream to the demethanizer column. An embodiment of the present disclosure is one, any or all of prior embodiments in this paragraph up through the first embodiment in this paragraph further comprising passing the demethanizer column bottoms liquid stream to the heat exchanger to provide a heat exchanged bottoms liquid stream; and passing the heat exchanged bottoms liquid stream to a deethanizer column. An embodiment of the present disclosure is one, any or all of prior embodiments in this paragraph up through the first embodiment in this paragraph wherein the olefin stream comprising C2 and/or C3 olefins is produced from reacting oxygenates over a SAPO catalyst. An embodiment of the present disclosure is one, any or all of prior embodiments in this paragraph up through the first embodiment in this paragraph further comprising passing the demethanizer column overhead vapor stream to the heat exchanger to heat exchange with the olefin stream and the refrigerant stream to provide a heat exchanged overhead vapor stream. An embodiment of the present disclosure is one, any or all of prior embodiments in this paragraph up through the first embodiment in this paragraph further comprising passing the demethanizer column bottoms liquid stream to the heat exchanger to heat exchange with the olefin stream and the refrigerant stream to provide a heat exchanged bottoms liquid stream. An embodiment of the present disclosure is one, any or all of prior embodiments in this paragraph up through the first embodiment in this paragraph further comprising passing the mixed refrigerant stream to a refrigerant compressor to provide a compressed mixed refrigerant stream; passing the compressed mixed refrigerant stream to the heat exchanger to provide the cooled olefin stream and a cooled refrigerant stream; and passing the cooled refrigerant stream to the refrigerant compressor. An embodiment of the present disclosure is one, any or all of prior embodiments in this paragraph up through the first embodiment in this paragraph further comprising expanding the cooled refrigerant stream to provide an expanded refrigerant stream; passing the expanded refrigerant stream to the heat exchanger to provide a heat exchanged refrigerant stream; and passing the heat exchanged refrigerant stream to the refrigerant compressor.

A second embodiment of the present disclosure is a process for separating an olefin stream from a methane stream comprising providing an olefin stream comprising C2 and/or C3 olefins; cooling the olefin stream in a heat exchanger with a mixed refrigerant stream to provide a cooled olefin stream; passing an overhead vapor olefin stream taken from the cooled olefin stream and a liquid olefin stream taken from the cooled olefin stream to a demethanizer column operating at an overhead pressure of about 344 kPa gauge (50 psig) to about 2069 kPa gauge (300 psig); fractionating the overhead vapor olefin stream and the liquid olefin stream in the demethanizer column to provide a demethanizer column overhead vapor stream, a demethanizer column bottoms liquid stream, and a first side stream; and passing the demethanizer column overhead vapor stream, the demethanizer column bottoms liquid stream, and the first side stream to the heat exchanger to heat exchange with the olefin stream and the refrigerant stream and provide a heat exchanged overhead vapor stream, a heat exchanged bottoms liquid stream, and a heat exchanged first side stream. An embodiment of the present disclosure is one, any or all of prior embodiments in this paragraph up through the second embodiment in this paragraph wherein the olefin stream is separated into a vapor olefin stream and a liquid olefin stream which are passed to the demethanizer column separately. An embodiment of the present disclosure is one, any or all of prior embodiments in this paragraph up through the second embodiment in this paragraph further comprises passing the vapor olefin stream to the heat exchanger; cooling the vapor olefin stream in the heat exchanger to provide a cooled vapor olefin stream; separating the cooled vapor olefin stream to provide the overhead vapor olefin stream and the bottom liquid olefin stream; and passing the overhead vapor olefin stream, the bottom liquid olefin stream, and the liquid olefin stream to the demethanizer column. An embodiment of the present disclosure is one, any or all of prior embodiments in this paragraph up through the second embodiment in this paragraph further comprising passing the first side stream to a reflux compressor to provide a compressed first side stream; passing the compressed first side stream to the heat exchanger; passing the heat exchanged first side stream to an overhead heat exchanger to provide a cooled reflux stream; and passing the cooled reflux stream to the demethanizer column. an embodiment of the present disclosure is one, any or all of prior embodiments in this paragraph up through the second embodiment in this paragraph wherein the step of passing the demethanizer column bottoms liquid stream to the heat exchanger comprises taking a second side stream and the demethanizer column bottoms liquid stream from the demethanizer column; passing the second side stream and the demethanizer column bottoms liquid stream to the heat exchanger to provide a heated side stream and a heat exchanged bottoms liquid stream; passing the heated side stream to the demethanizer column; and passing the heat exchanged bottoms liquid stream to a deethanizer column. An embodiment of the present disclosure is one, any or all of prior embodiments in this paragraph up through the second embodiment in this paragraph wherein the deethanizer column is in downstream fluid communication with the demethanizer column. An embodiment of the present disclosure is one, any or all of prior embodiments in this paragraph up through the second embodiment in this paragraph wherein the olefin stream comprising C2 and/or C3 olefins is produced from reacting oxygenates over a SAPO catalyst.

Without further elaboration, it is believed that using the preceding description that one skilled in the art can utilize the present disclosure to its fullest extent and easily ascertain the essential characteristics of the present disclosure, without departing from the spirit and scope thereof, to make various changes and modifications of the present disclosure and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

1. A process for separating an olefin stream from a methane stream comprising:
providing an olefin stream comprising C2 and/or C3 olefins;
cooling said olefin stream in a heat exchanger with a mixed refrigerant stream to provide a cooled olefin stream;
passing the cooled olefin stream to a demethanizer column operating at an overhead pressure of about 344 kPa gauge (50 psig) to about 2069 kPa gauge (300 psig); and
fractionating said cooled olefin stream in the demethanizer column to provide a demethanizer column overhead vapor stream and a demethanizer column bottoms liquid stream.

2. The process of claim 1 wherein said olefin stream is separated into a vapor olefin stream and a liquid olefin stream which are passed to the demethanizer column separately.

3. The process of claim 2 further comprises:
passing said vapor olefin stream to the heat exchanger;
cooling said vapor olefin stream in the heat exchanger to provide a cooled vapor olefin stream;
separating said cooled vapor olefin stream to provide an overhead vapor olefin stream and a bottom liquid olefin stream; and
fractionating said overhead vapor olefin, said bottom liquid olefin stream, and said liquid olefin stream in the demethanizer column.

4. The process of claim 1 further comprising:
taking a first side stream from the demethanizer column;
passing said first side stream to the heat exchanger to provide a heat exchanged first side stream; and
passing said heat exchanged first side stream to the demethanizer column.

5. The process of claim 4 further comprising:
passing said first side stream to a reflux compressor to provide a compressed first side stream;
passing said compressed first side stream to the heat exchanger to provide a heat exchanged first side stream;
passing said heat exchanged first side stream to the demethanizer column.

6. The process of claim 5 further comprising:
passing said heat exchanged first side stream to an overhead heat exchanger to provide a cooled reflux stream;
expanding said cooled reflux stream to provide an expanded reflux stream; and
passing said expanded reflux stream to the demethanizer column.

7. The process of claim 1 further comprising:
taking a second side stream from the demethanizer column;
passing said second side stream to the heat exchanger to provide a heated second side stream; and
passing said heated second side stream to the demethanizer column.

8. The process of claim 1 further comprising:
passing said demethanizer column bottoms liquid stream to the heat exchanger to provide a heat exchanged bottoms liquid stream; and
passing said heat exchanged bottoms liquid stream to a deethanizer column.

9. The process of claim 1 wherein said olefin stream comprising C2 and/or C3 olefins is produced from reacting oxygenates over a SAPO catalyst.

10. The process of claim 1 further comprising passing said demethanizer column overhead vapor stream to the heat exchanger to heat exchange with said olefin stream and said refrigerant stream to provide a heat exchanged overhead vapor stream.

11. The process of claim 1 further comprising passing said demethanizer column bottoms liquid stream to the heat exchanger to heat exchange with said olefin stream and said refrigerant stream to provide a heat exchanged bottoms liquid stream.

12. The process of claim 1 further comprising:
passing said mixed refrigerant stream to a refrigerant compressor to provide a compressed mixed refrigerant stream;
passing said compressed mixed refrigerant stream to the heat exchanger to provide said cooled olefin stream and a cooled refrigerant stream; and
passing said cooled refrigerant stream to the refrigerant compressor.

13. The process of claim 12 further comprising:
expanding said cooled refrigerant stream to provide an expanded refrigerant stream;
passing said expanded refrigerant stream to the heat exchanger to provide a heat exchanged refrigerant stream; and
passing said heat exchanged refrigerant stream to the refrigerant compressor.

14. A process for separating an olefin stream from a methane stream comprising:
providing an olefin stream comprising C2 and/or C3 olefins;
cooling said olefin stream in a heat exchanger with a mixed refrigerant stream to provide a cooled olefin stream;
passing an overhead vapor olefin stream taken from said cooled olefin stream and a liquid olefin stream taken from said cooled olefin stream to a demethanizer column operating at an overhead pressure of about 344 kPa gauge (50 psig) to about 2069 kPa gauge (300 psig);
fractionating said overhead vapor olefin stream and said liquid olefin stream in the demethanizer column to provide a demethanizer column overhead vapor stream, a demethanizer column bottoms liquid stream, and a first side stream; and
passing said demethanizer column overhead vapor stream, said demethanizer column bottoms liquid stream, and said first side stream to the heat exchanger to heat exchange with said olefin stream and said refrigerant stream and provide a heat exchanged overhead vapor stream, a heat exchanged bottoms liquid stream, and a heat exchanged first side stream.

15. The process of claim 14 wherein said olefin stream is separated into a vapor olefin stream and a liquid olefin stream which are passed to the demethanizer column separately.

16. The process of claim 15 further comprises:
passing said vapor olefin stream to the heat exchanger;
cooling said vapor olefin stream in the heat exchanger to provide a cooled vapor olefin stream;
separating said cooled vapor olefin stream to provide said overhead vapor olefin stream and said bottom liquid olefin stream; and
passing said overhead vapor olefin stream, said bottom liquid olefin stream, and said liquid olefin stream to the demethanizer column.

17. The process of claim 14 further comprising:
passing said first side stream to a reflux compressor to provide a compressed first side stream;
passing said compressed first side stream to the heat exchanger;
passing said heat exchanged first side stream to an overhead heat exchanger to provide a cooled reflux stream; and
passing said cooled reflux stream to the demethanizer column.

18. The process of claim 14 wherein the step of passing said demethanizer column bottoms liquid stream to the heat exchanger comprises:
taking a second side stream and said demethanizer column bottoms liquid stream from the demethanizer column;
passing said second side stream and said demethanizer column bottoms liquid stream to the heat exchanger to provide a heated side stream and a heat exchanged bottoms liquid stream;
passing said heated side stream to the demethanizer column; and
passing the heat exchanged bottoms liquid stream to a deethanizer column.

19. The process of claim 18 wherein the deethanizer column is in downstream fluid communication with the demethanizer column.

20. The process of claim 14 wherein said olefin stream comprising C2 and/or C3 olefins is produced from reacting oxygenates over a SAPO catalyst.
